(19) United States
(12) Patent Application Publication
(10) Pub. No.: US 2026/0020941 A1
(43) Pub. Date: Jan. 22, 2026

(54) METHOD FOR SCANNING A DENTAL ARCH OF A USER

(71) Applicant: DENTAL MONITORING, Paris (FR)

(72) Inventors: Thomas PELLISSARD, Maisons-Alfort (FR); Guillaume GHYSELINCK, CANTIN (FR); Xavier DOREMUS, MONTROUGE (FR)

(21) Appl. No.: 19/106,498

(22) PCT Filed: Aug. 29, 2023
(86) PCT No.: PCT/EP2023/073628

§ 371 Date: Feb. 25, 2025

(30) Foreign Application Priority Data
Aug. 31, 2022 (FR) FR2208709

(51) Int. Cl. A61C 9/00; A61B 1/24; G06V 20/40; G06V 20/70

(52) U.S. Cl. CPC A61C 9/0053; G06V 20/46; G06V 20/70; A61B 1/24; G06V 2201/03

(57) ABSTRACT

A method for scanning a dental arch of a user, the method comprising:

a prior step of generating a reference representation, then-a plurality of cycles of the following steps a) to d): a) acquisition of an updated image by the user by means of an image acquisition apparatus; b) analysis of the updated image in order to identify the surface of the updated image that represents the dental arch of the user, referred to as “acquired 2D surface”, then outlining and labeling a surface of the reference representation that corresponds to the acquired 2D surface, or “elementary labeled region”, and adding the elementary labeled region to a “labeled region of the reference representation” that brings together all the elementary labeled regions; c) determining and presenting guide information for guiding towards new acquisition conditions; d) preferably moving the acquisition apparatus towards said new acquisition conditions.

### TECHNICAL FIELD

The present invention relates to a method for scanning a dental arch of a user, and a device for implementing such a method.

### PRIOR ART

A scan of the dental arches of a user is traditionally performed to analyze the dental situation, particularly before orthodontic treatment is established. However, performing a scan of the dental arches of a user requires the user to visit a dental care professional or to transmit information, in particular images, to the dental care professional. Performing a scan in a dental office, or more generally from a dental care professional, may however entail not only significant costs but also stress for the user. In addition, the user's own acquisition of dental images can lead to insufficient image quality or incomplete content.

There is a need to facilitate the acquisition of dental arch scans, while limiting the risk of incomplete scans.

One aim of the invention is to meet this need.

### DISCLOSURE OF THE INVENTION

### Summary Of The Invention

The invention provides a method for scanning a dental arch of a user, the method comprising
a prior step of determining a model of a reference dental arch, referred to as the “reference representation”, then
a plurality of cycles of the following steps a) to d):
   a) acquisition, under acquisition conditions, of a two-dimensional image of said dental arch, or “updated image”, by means of an image acquisition apparatus, preferably by the user;
   b) analysis of the updated image so as to identify the surface of the updated image representing the dental arch of a user, referred to as the “acquired 2D surface”, then outlining and labeling a surface of the reference representation that corresponds to the acquired 2D surface, referred to as the “elementary labeled region”, and adding the elementary labeled region to a “labeled region of the reference representation” that brings together all the elementary labeled regions defined during the previous cycles, which were empty prior to the first of said cycles;
   c) determination and presentation to the user of guide information for guiding the acquisition apparatus towards new acquisition conditions suitable for the next cycle to increase the labeled region of the reference representation;
   d) moving the acquisition apparatus towards said new acquisition conditions; the method preferably comprising, after the last cycle, a final step of generating a final three-dimensional model from said updated images.

The generating of the final model can be carried out by the image acquisition apparatus or by a computer remote from the image acquisition apparatus and to which the set of acquired updated images has been transmitted, preferably to a dental care professional's computer.

As will be seen in greater detail later in the description, a scanning method according to the invention quickly and easily leads to a set of updated images sufficient to produce a complete final model of the dental arch.

Furthermore, there is no need for the user to visit a dental care professional, or for the user to be supervised by a dental care professional to acquire updated images. With the guiding, the updated images can advantageously be acquired under precise acquisition conditions, without any special training.

Advantageously, acquisition can be carried out in real time, with updated images extracted from a video, for example.

Preferably, in step c), the guide information is presented on a screen, preferably on a screen of the image acquisition apparatus. In particular, the image acquisition apparatus can be a cell phone or a tablet, preferably a cell phone.

In particular, the guide information may comprise audio, visual and/or sensory information. In this way, guide information can be tailored to any user. Preferably, the guide information comprises several different types of information, preferably stimulating several different senses, thus facilitating communication to the user.

In a preferred embodiment of the invention, the labeling comprises, for each step b), a visual labeling of the surface of the reference representation, and each step c) comprises presenting, preferably to the user, preferably on a screen, preferably on a screen of a cell phone of the user used to acquire the updated images, all the visual labels generated in all the preceding steps b).

A visual label preferably comprises a graphic representation of the elementary labeled region, for example a representation of the outline of the elementary labeled region and/or a solid color and/or pattern within the boundaries of the elementary labeled region.

This simplifies acquisition. In particular, during real-time cycling, the screen displays a reference representation whose labeled region is completed as the cycles progress, enabling the user to easily identify the unlabeled region, and to position and orient the image acquisition apparatus accordingly.

Conversely, during real-time cycling, the display can show a reference representation, with the unlabeled region fading as the cycles progress. Preferably, however, the screen continues to represent at least the contours of the dental arch.

The visual labels are therefore a preferred form of guide information.

The guide information may comprise additional information, the additional information corresponding to information other than that intended to indicate the positioning of the acquisition apparatus, the additional information may, for example, indicate to the user to put in or remove an orthodontic aligner, to open or close the mouth, and/or to use a retractor or, on the contrary, to remove it.

The reference representation can be generated. Alternatively, it can be selected, for example from a database.

The reference representation can be two-dimensional. In a preferred embodiment, the reference representation is a digital three-dimensional model. By acquiring different updated images, the method marks different areas of this model until a model coverage threshold is reached, or preferably until the model is completely labeled. The updated images then advantageously “cover” a majority of the model surface, or preferably the entire model surface, and enable, for example, the production of a final model representing the dental arch of a user with a high degree of precision. The final model can be produced by the dental care professional's computer, to which the updated images are transmitted, or by the image acquisition apparatus.

In particular, the coverage threshold can be 90%, better still 95%, even better still 99%.

At the end of each cycle, the method may comprise comparing the percentage of the labeled region's surface area as a share of the model's total surface area, known as the “coverage percentage”, with the coverage threshold. If the coverage percentage is above the threshold, the final model generation step is performed, otherwise a new cycle is started.

In a particular embodiment, in step c), the coverage percentage is presented to the user.

The presentation of the coverage percentage can take the form of a gauge, a numerical display, a progress bar, objectives presented successively when a particular objective is reached, or simultaneously and presenting a different display that distinguishes objectives reached from those still to be reached. For example, the objectives are “acquisition of molars”, “acquisition of incisors”, “acquisition of more than 10% of the model”, “acquisition of more than 25% of the model”, “acquisition of more than 40% of the model”, “acquisition of more than 50% of the model”, “acquisition of more than 75% of the model”, “acquisition of more than 85% of the model”, “acquisition of right view”, “acquisition of left view”, “acquisition of top view”, “acquisition of bottom view”; “acquisition of a closed-mouth view”, “acquisition of an open-mouth view”, “acquisition with an orthodontic aligner”.

A method according to the invention may furthermore particularly comprise one or more of the following optional features:
the updated images of successive cycles are extracted from a video acquired by the user via a tablet or a cell phone, said cycles preferably being executed in real time during the acquisition of said video, at least part of the guide information preferably being presented on a screen of said tablet or said phone, respectively;
the guide information comprises a presentation of the labeled region on a screen of the image acquisition apparatus, preferably superimposed on the reference representation, wherein the unlabeled region has an appearance, preferably a color, different from the labeled region;
the reference representation is a “reference” model of a “reference” arch
representative of the “historic” arches of a set comprising more than 10, more than 100, more than 1,000, more than 10,000 and/or less than 1,000,000 historic individuals, or
formed by assembling tooth models in an arrangement determined by analysis of one or more analysis images of the dental arch of a user, preferably acquired with the image acquisition apparatus
one or more illustrations, in 3D or 2D, of a dental arch, possibly photorealistic;
in step c) of any cycle, a view of the observed reference model is presented along a direction of observation oriented, with respect to the reference model, like the direction of acquisition of the image updated in step a) of said cycle, with respect to the dental arch of the user, that is substantially oriented like the direction of the optical axis of the camera during said acquisition, so that said view is substantially superimposable in register with said updated image;
in a step c), preferably in any step c), the image acquisition apparatus provides variable information as a function of a difference between the actual acquisition conditions and said new acquisition conditions, for example by emitting a sound signal or a visual indication, e.g. a flashing light, the more intense said differences are; the user thus advantageously knows whether the movement of the image acquisition apparatus which he performs is more effective in reaching the new acquisition conditions or less so;
for step a) of at least one cycle, preferably for any step a), the lips and/or cheeks of the dental arch of a user are spread apart, preferably using a retractor, so as to expose at least one tooth to the image acquisition apparatus, for example to completely expose the outer surface of an incisor and/or at least partially expose the outer surface of a molar;
during the cycles, the following are acquired:
at least one updated image taken facing the user and/or at least one updated image taken to the right of the user and/or at least one updated image taken to the left of the user, preferably at least one updated image taken facing the user and at least one updated image taken to the right of the user and at least one updated image taken to the left of the user; and/or
at least one updated image taken facing the user, on the one hand, and at least one updated image taken from above the user and/or at least one updated image taken from below the user, on the other hand; preferably at least one updated image taken facing the user and at least one updated image taken from above the user and at least one updated image taken from below the user, and/or
at least one updated image taken with the mouth open and/or at least one updated image taken with the mouth closed;
the user is at a distance, preferably more than 100 m or more than 1 km and/or less than 1000 km from any dental care professional, in particular any orthodontist;
the cycles are stopped when more than 50%, more than 70%, more than 90%, preferably 100% of the surface of the reference representation is “covered” by the acquired updated images, that is depicted in at least one updated image;
the final model is compared with another model representing the dental arch of a user, for example to correct the final model or the other model, and/or the final model is broken down into tooth models and, preferably, said tooth models are moved;
the final model is compared with the reference model, and this comparison is preferably presented to the user, for example by superimposing the final model and the reference model, preferably in alignment;
the reference representation is a three-dimensional model and the surface of the reference representation corresponding to the acquired 2D surface is determined
by searching for suitable virtual acquisition conditions for acquiring a view of the reference representation, referred to as the “reference image”, that best matches the image updated under said virtual acquisition conditions; then
by projecting the acquired 2D surface onto the reference representation, along the direction of observation of said reference representation under said virtual acquisition conditions;
the virtual acquisition conditions are determined by seeking to superimpose noteworthy points of the dental arch represented on the updated image with the corresponding noteworthy points of the reference representation.

The invention further relates to:
a computer program, and in particular specialized software for cell phones or tablets, or an “app”, comprising program code instructions for executing one or more steps of a method according to the invention, preferably for all steps b) to c) when said program is executed by a computer,
a data medium on which such a program is recorded, for example a memory or a CD-ROM.

Preferably, when the computer program is specialized software for cell phones or tablets, the computer program comprises instructions for executing step a), and even more preferably for executing all steps a) to c).

The invention further relates to a device for implementing a method according to the invention, said device comprising:
optionally, a first computer having a first computer program comprising program code instructions for generating the reference representation or selecting the reference representation, for example from a database;
an image acquisition apparatus for carrying out steps a);
preferably a screen for displaying guide information during steps c);
a second computer, the same as or different from the first computer, comprising a second computer program comprising program code instructions for executing steps b) and c);
a third computer, the same as or different from the first and/or second computer(s), comprising a third computer program comprising program code instructions for implementing the final step of generating the final model of the dental arch of a user from the updated images;
preferably a fourth computer, the same as or different from the first and/or second and/or third computer(s), comprising a fourth computer program comprising program code instructions for comparing the final model with another model representing the dental arch of a user and/or breaking down the final model into tooth models and, preferably, moving said tooth models.

Preferably, the screen and/or the first computer and/or the second computer and/or the third computer and/or the fourth computer is/are integrated into the acquisition apparatus. Preferably, the image acquisition apparatus is a cell phone or tablet. Preferably, the screen is a cell phone or tablet screen, respectively.

The user can then easily, without the intervention of a third party, and in particular without the intervention of a dental care professional, by means of a simple cell phone or tablet, acquire dental images of good quality that collectively fully represent the reference representation.

The device may further comprise communication means, in particular for sending the updated image(s) and/or receiving the reference representation.

### Definitions

A “user” is a person for whom a method according to the invention is implemented.

The term “dental care professional” refers to any person qualified to provide dental care, including orthodontists and dentists.

The “mouth closed” position is the occlusion position wherein the user's upper and lower teeth are in contact. An “open mouth” position is one wherein the user's upper and lower teeth are not in contact, preferably the position with the mouth fully open.

An “arch” or “dental arch” means all or part of a dental arch, preferably comprising at least 2, preferably at least 3, more preferably at least 4 teeth.

A retractor (or dental retractor) is a device used to pull back the lips. It comprises an upper and a lower flange, and/or a right and a left flange, extending around a retractor opening and intended to be inserted between the teeth and the lips. In the operating position, the user's lips rest on these edges, so that the teeth are visible through the retractor opening. A retractor thus makes it possible to observe the teeth without being obstructed by the lips. However, the teeth do not rest on the retractor, so that by turning the head relative to the retractor, the user can change the teeth that are visible through the retractor opening. The user can also change the spacing between their dental arches. In particular, a retractor does not press on the teeth to spread the two jaws apart, but rather on the lips. In one embodiment, a retractor is configured to elastically spread the upper and lower lips apart to expose the teeth visible through the retractor opening. In one embodiment, a retractor is configured so that the distance between the top edge and the bottom edge, and/or between the right edge and the left edge, is constant. Retractor are described, for example, in PCT/EP2015/074896, U.S. Pat. No. 6,923,761, or US 2004/0209225.

According to the international convention of the FDI World Dental Federation, each tooth in a dental arch has a predetermined number.

A “noteworthy point” is a point on an arch, tooth, arch model, or tooth model that can be identified, e.g. the apex of the tooth or at the tip of a cusp, a point of interdental contact, that is, of a tooth with an adjacent tooth, e.g. a mesial or distal point of the incisal edge of a tooth, or a point at the center of the tooth crown, or “barycenter”.

By “computer” we mean a computer processing unit, which includes a set of several machines with computer processing capabilities. In particular, this unit can be integrated into a cell phone, or be a PC-type computer or server, for example a server remote from the user, e.g. being the “cloud” or a computer located at a dental care professional's premises. The cell phone and the computer then have the means to communicate with each other.

Typically, a computer comprises a processor, a memory, a human-machine interface, typically comprising a screen, and a communication module via the Internet, WIFI, Bluetooth® or the telephone network. Software configured to implement a method of the invention is loaded into the computer's memory. The computer can also be connected to a printer.

The method according to the invention (other than the operations of acquisition and of moving the image acquisition apparatus) is implemented by computer, preferably exclusively by computer.

The term “model” means a three-dimensional digital model. A model is made up of a set of voxels.

A “tooth model” is a three-dimensional digital model of a tooth. A dental arch model can be cut to define tooth models for at least some, preferably all, of the teeth represented in the arch model. Tooth models are therefore models within the arch model.

An “arch model” is a model representing at least part of a dental arch, preferably at least 2, preferably at least 3, most preferably at least 4 teeth.

The “breakdown” of an arch model into “tooth models” is an operation that delimits and makes autonomous the tooth representations (tooth models) in the arch model. Computer tools are available to manipulate tooth models in an arch model. An example of software for manipulating tooth models and creating a treatment scenario is the program Treat, described at https://en.wikipedia.org/wiki/Clear_aligners #cite_note-invisalignsystem-10.

An “image” refers to a two-dimensional image, such as a photograph or a video frame. An image is made up of pixels. A “video” is considered to be a collection of photos.

“Image of an arch”, or “model of an arch”, or “reference representation” of a dental arch means a representation of all or part of said arch, preferably representing at least 2 teeth, more preferably at least 4 teeth.

The “match” or “fit” between two objects, for example between two images of a dental arch, is a measure of the difference, or “distance”, between them. A “best fit” is achieved when this difference is minimal, in particular when the two images represent the same elements in essentially the same way, that is in such a way that the element representations on these two images are essentially superimposable in alignment.

A superimposition of two images is “in alignment” when the contours of representations of physical elements in the first image are superimposed on the contours of said physical elements in the second image. The superimposition thus appears realistic.

“Labeling” a surface with a reference representation consists in storing sufficient data in computer memory to specifically reconstruct that surface. In particular, the boundaries of this surface can be represented on the reference representation so that the user can visualize it. The labeling is then described as “visual”.

The “acquisition conditions” of an image specify the position and orientation in space of a device for acquiring this image relative to the user's teeth (real acquisition conditions) or to a model of the user's teeth (virtual acquisition conditions), and preferably the calibration of this acquisition apparatus. Acquisition conditions are said to be “virtual” or “theoretical” when they correspond to a simulation wherein the acquisition apparatus would be in said acquisition conditions (theoretical positioning and preferably calibration of the acquisition apparatus) relative to a model.

Particularly under virtual acquisition conditions of a reference image, the acquisition apparatus can also be described as “virtual”. The image is in fact acquired by a fictitious acquisition apparatus, having the characteristics of the “real” acquisition apparatus that was used to acquire the real images, and in particular the updated images.

The “calibration” of an acquiring apparatus is made up of all the calibration parameter values. A calibration parameter is a parameter intrinsic to the acquisition apparatus (unlike its position and orientation) whose value influences the acquired image. Preferably, calibration parameters are chosen from the group formed by aperture, exposure time, focal length and sensitivity.

A “statistical treatment” is one which, when applied to a set of data, enables us to determine characteristics specific to this set, such as a mean, a standard deviation, or a median value. Statistical processing tools are well known to the person skilled in the art.

An “illustration” has no physical reality. It may be a symbolic or schematic representation comprising a set of geometric shapes, or a 2D or 3D digital drawing.

“First”, “second”, “third” and “fourth”, “reference”, “updated”, “historical”, “target” are used for clarity.

“Vertical”, “horizontal”, “right”, “left”, “in front” or “from the front”, “behind”, “above”, “below” refer to a user standing vertically.

Unless otherwise indicated, “including” or “comprising” or “having” should be interpreted in a non-restrictive manner.

### BRIEF DESCRIPTION OF THE FIGURES

Further features and advantages of the invention will become apparent from the following detailed description and from an examination of the appended drawing, wherein:

FIG. 1 schematically shows an example of a cycle of a method according to the invention;

FIG. 2 schematically shows an example of a device according to the invention;

FIG. 3 shows the progressive coloring of a reference model.

### DETAILED DESCRIPTION

### Method

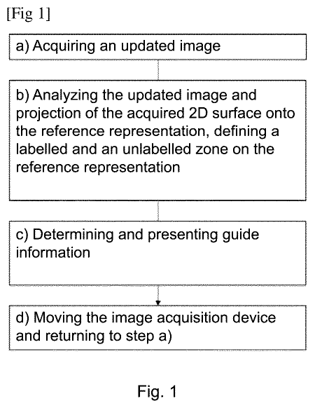

A method of scanning a dental arch of a user according to the invention comprises steps a) to d) as shown in FIG. 1.

Preferably, it further comprises a prior step of determining a reference representation.

Determining the reference representation can involve generating a representation, in particular a three-dimensional model.

In another embodiment, determining the reference representation may consist in selecting a representation from a database.

The reference representation is a model or image representing a reference dental arch. Preferably, the reference representation is a model, called a “reference model”.

The reference representation may represent a standard reference dental arch, and can be used for different users, possibly for any user. However, the reference representation preferably represents a reference dental arch whose shape is close to that of the user, which improves the accuracy of the method.

The reference representation can be representative of a population of individuals.

In one embodiment, the reference representation is obtained by statistical processing of a set of historical arch representations of a population of historical individuals having one or more characteristics in common with the user, for example grouping together people of an age class identical to that of the user or afflicted with the same pathology as the user. Statistical processing may, for example, consist of determining a reference representation that minimizes differences in shape with historical arch representations.

In particular, the reference representation can be a typodont model.

The reference representation can be obtained by arranging historical tooth models in an arrangement corresponding to one or more “analysis” images of the user's arch, preferably taken with the user's cell phone. In particular, it is possible to proceed as follows:
creating a historical library with more than 1,000, preferably more than 10,000 and/or less than 1,000,000 tooth models, so-called “historical tooth models”, and preferably assigning a tooth number to each historical tooth model;
analysis of at least one “analysis image” of the dental arch, for example by means of a deep learning device, preferably a neural network, so as to determine at least one region of the analysis image representing a tooth, or “tooth analysis region”, and preferably the number of said tooth;
for each tooth analysis region determined in the previous step, searching the historical library for a historical tooth model having maximum proximity in shape to the tooth analysis region, or “optimal tooth model” and preferably having the same tooth number as the tooth depicted in the tooth analysis region;
arranging the set of optimal tooth models to create a dental arch model with maximum proximity to the arch depicted in the analysis image, or “assembled model”;
optionally, replacing at least one optimal tooth model with another tooth model and repeating the previous step so as to maximize the match between the assembled model and the analysis image;
optionally, repeating said analysis with another analysis image and, in the step of arranging and/or replacing, searching for a best fit for the set of analysis images used.

A method for generating a reference representation in the form of a model by arranging historical tooth models is described in particular in European application no. 18 184486.

The reference representation can be generated by the image acquisition apparatus, in particular from images of the dental arch of a user taken with the image acquisition apparatus. It may be generated by another apparatus, such as the dental care professional's computer.

The reference representation can be stored in a memory or a cloud, accessible via digital communication means. It is made accessible to the computer that carries out steps b) and c).

After the reference representation has been made available, the method comprises a plurality of cycles of steps a) to d).

The time interval between two successive cycles is preferably less than 5 minutes, 1 minute, 30 seconds or 1 second. Preferably, the user acquires updated images in real time, preferably by taking video of his dental arch, and steps b) to d) are immediately carried out for each updated image. The reference representation is thus labeled, preferably visually, in real time, and the user is guided through the process of making the video.

Preferably, a cycle is performed for at least one updated image taken facing the user, at least one updated image taken to the right of the user and at least one updated image taken to the left of the user.

Preferably, a cycle is performed for at least one updated image taken with the mouth open and for at least one updated image taken with the mouth closed.

In the first cycle, the reference representation is partially labeled in step b). The reference representation labeling is then completed for each subsequent cycle.

An example cycle is now described in detail.

In step a), an updated image, preferably a photo, depicting a dental arch of a user, is acquired using an image acquisition apparatus. Preferably, a video is acquired with the image acquisition apparatus, and the updated image is extracted from the video.

In a particular embodiment, a step a) comprises the acquisition of a plurality of updated images, preferably photos, representing a dental arch of a user, by means of an image acquisition apparatus, the plurality of images being extractable from a video.

Preferably, acquisition is carried out by the user on their own.

The updated image is preferably “extraoral”, that is without the camera lens being inserted into the user's mouth.

The image acquisition apparatus can in particular be a cell phone, a tablet, a camera or a computer, the image acquisition apparatus preferably being a cell phone or a tablet, in particular so that the user can acquire up-to-date images anywhere, and in particular outside a dental care professional's office, for example more than 1 km from a dental care professional's office.

In one embodiment, the user uses a cell phone and a holder to which the cell phone is removably attached, the holder being held against the user during the acquisition of any updated images. In particular, the holder may be in PCT/EP2021/068702, EP17306361, of the type described PCT/EP2019/079565, PCT/EP2022/053847 or FR2113577.

In a preferred embodiment, the user uses a free cell phone, that is one whose position and orientation can be freely determined, and in particular not attached to a holder. In fact, the method described in the invention enables the user to be guided in taking images, so that guidance by means of a holder is not essential. Preferably, the image acquisition apparatus is not in contact with the user's mouth, either directly or via a holder for the image acquisition apparatus.

The time interval between the prior step of generating the reference representation and step a) is preferably less than 4 weeks, 2 weeks or 1 week, in particular when said generating is performed on the basis of analysis images of the dental arch of a user. The prior step can also be performed immediately before step a), for example less than 5 minutes before step a).

In step b), the updated image is analyzed to identify, in the updated image, a surface at least partially depicting the dental arch of a user, referred to as the “acquired 2D surface”.

When a plurality of updated images are acquired in step a) preceding step b), each updated image of the plurality of updated images is analyzed to identify an acquired 2D surface in each updated image.

Analysis can be performed using conventional segmentation methods.

A search then takes place for a part of the surface of the reference representation that corresponds to the acquired 2D surface. Preferably, said part results from a one-to-one relationship with the acquired 2D surface, that is, for each acquired 2D surface, a single part of the surface of the reference representation is defined.

Preferably, said part is the result of a projection of the acquired 2D surface onto the reference representation.

In a preferred embodiment, the reference representation is a model. We then search for a model view that matches the updated image, that is which represents the modeled arch in much the same way as the updated image. In other words, virtual acquisition conditions are sought that are suitable for acquiring a view of the model that best fits the image updated under said virtual acquisition conditions. These virtual acquisition conditions can be determined by searching for virtual acquisition conditions that superimpose noteworthy points on the updated image onto corresponding noteworthy points on the model, preferably by means of an optimization algorithm.

A search for a model view that matches the updated image is described, for example, in European application no. 18 184486.

Said view and the updated image can then be superimposed substantially in alignment on each other. By superimposing the model view and the updated image in alignment, it is possible to define a region of the view that is covered by, or covers, the acquired 2D surface. The boundaries of this region can be transferred to the model. Once they have been, they outline a part of the surface of the reference representation that corresponds to the acquired 2D surface. Said part is then the result of a projection of the acquired 2D surface onto the reference representation along the model viewing direction from which the view represents the model.

Said part of the reference representation surface corresponding to the acquired 2D surface is then labeled. This is known as the “elementary labeled region”. A “labeled region” is the union of all the elementary labeled regions defined during the cycles. The labeled region, initially empty before the first cycle, thus comprises an elementary labeled region at the end of the first cycle, and is then completed at each cycle, as the acquisition apparatus moves and updated images are acquired. The “unlabeled region”, representing the parts of the reference representation that correspond to regions of the dental arch that do not appear on any updated image, is therefore gradually reduced.

In step c), image acquisition apparatus guide information is determined and presented.

The guide information is useful for helping the user to move the image acquisition apparatus so that, during the next cycle, the acquired updated image represents a new acquired 2D surface which enables the labeled region to be completed, that is which leads to an elementary labeled region at least partly outside the labeled region defined at the end of step b) of the current cycle.

The guide information informs the user of the parts of his dental arch for which that person still needs to acquire one or more updated images. It guides him to orient and/or position the acquisition apparatus to achieve the desired acquisition.

In one embodiment, the computer implementing step c) determines, for example by a random search or with an optimization algorithm, said new acquisition conditions, for the next cycle, so that the new 2D surface acquired under these new acquisition conditions maximizes the increase in the labeled region of the reference representation. These new acquisition conditions can be described as new “target” acquisition conditions.

The guide information may comprise visual information, and/or audible information, and/or sensitive information. For example, the guide information may comprise audio instructions telling the user to move the acquisition apparatus towards or away from the teeth, to shift the acquisition apparatus to the right or left, to rotate the acquisition apparatus around the dental arch, to open or close the mouth, or to open or close the jaw.

Sensitive information can be a vibration, for example, indicating to the user to stop a movement.

Preferably, in step c), visual guide information is presented, preferably on a screen, preferably on a screen of the image acquisition apparatus, preferably on a cell phone or tablet screen.

In one embodiment, the labeled region and, preferably, the unlabeled region are displayed. This display helps to guide the user quickly and efficiently. This guidance system, which gives the user a great deal of freedom, is intuitive, so no prior training is required.

The real-time cycles make it possible to see immediately how the labeled region is expanding. The gradual coloring of the reference representation is enjoyable. In one embodiment, a goal can be set for the user, for example, to encourage them to color the reference representation as quickly as possible, further enhancing the enjoyable aspect of acquiring updated images.

Preferably, the labeled region is colored, preferably a different color from the unlabeled region. Preferably, the contours of elementary labeled regions are not specifically represented, with only the contour of the labeled region possibly being shown. The labeling in step b) is then, preferably, a visual labeling consisting of extending the colored labeled region so that it incorporates the elementary labeled region defined in said step b).

By coloring the labeled region, and preferably by displaying the unlabeled region, the user is indirectly instructed how to move the image acquisition apparatus so as to acquire, during the next cycle, an updated image representing a part of the dental arch corresponding to an uncolored area and therefore to an unlabeled region of the reference representation.

In the same way as progressive coloring of the labeled region, it is possible to progressively erase the unlabeled region. On the screen, these two solutions are similar, the deletion of a region resulting in its being colored in the background color.

The guide information can be determined according to the acquisition conditions of the updated image acquired in the previous step a) and the unlabeled region.

Determining the guide information as a function of the unlabeled region, or equivalently, the labeled region, means that the guide information is not solely dependent on the position of the image acquisition apparatus in space. It depends on what the image acquisition apparatus has observed up to that point, and therefore not only on the position of the image acquisition apparatus in space, but also on the orientation of the image acquisition apparatus lens in relation to the dental arch during image acquisition.

A representation of the trajectory of the image acquisition apparatus, as described in US2020193871A1, does not depend on the labeled region, but only on the position of the image acquisition apparatus in space.

Steps b) and c) are implemented by a computer program.

In step d), the user modifies the position and/or orientation of the acquisition apparatus according to the guide information.

Preferably, the cycle of steps a) to d) is repeated until a stopping criterion is reached.

The stopping criterion can be a ratio of the labeled surface area to the surface area of the reference representation. In particular, steps a) to d) can be repeated until said ratio is greater than 50%, preferably greater than 75%, more preferably greater than 80%, more preferably greater than 90%, more preferably greater than 95%, more preferably 100%, that is until the reference representation no longer contains any unlabeled regions.

Finally, the scanning method according to the invention comprises, after stopping the cycling from a) to d), a step for generating a 3D model from the updated images acquired during the various cycles.

Preferably, after cycling through steps a) to d), the method comprises a step of sending the updated images acquired during the various cycles and/or a model generated from the updated images acquired during the various cycles, preferably to a dental care professional.

In particular, the model can be used by a dental care professional to diagnose or design an orthodontic treatment plan, or even to manufacture an orthodontic appliance, preferably an orthodontic aligner.

### Device

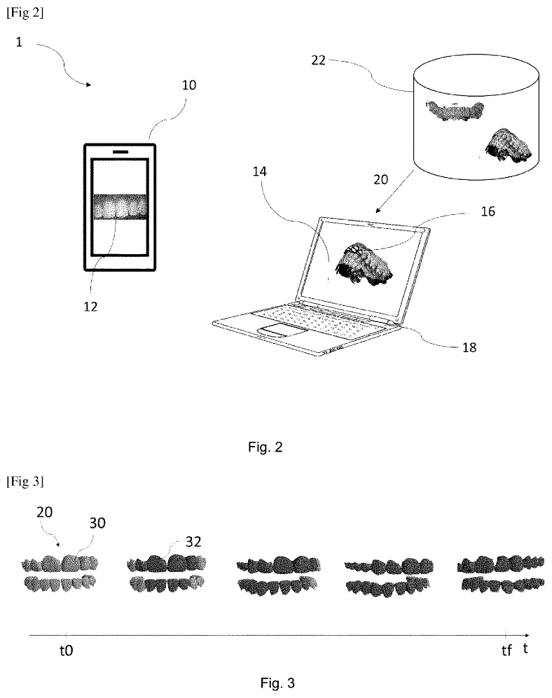

The invention further relates to a device for implementing a scanning method according to the invention. Such a device is shown in FIG. 2.

A device 1 according to the invention comprises:
optionally, a first computer comprising a first computer program comprising program code instructions for generating the reference representation;
an image acquisition apparatus 10 for carrying out steps a) to acquire updated images 12,
a display 14 for presenting guide information 16 during steps c),
a second computer 18 having a second computer program comprising program code instructions for executing steps b) and c), the second computer 18 receiving a reference model 20 stored in a database 22,
preferably, a third computer, the same as or different from the first and/or second computer(s), comprising a third computer program comprising program code instructions for generating a final model of the dental arch of a user from the updated images;
preferably a fourth computer, the same as or different from the first and/or second and/or third computer(s), comprising a fourth computer program comprising program code instructions for comparing the final model with another model representing the dental arch of a user and/or breaking down the final model into tooth models and, preferably, moving said tooth models.

Preferably, the screen and/or the first computer and/or the second computer and/or the third computer and/or the fourth computer is/are integrated into the image acquisition apparatus.

Preferably, the image acquisition apparatus is a cell phone or tablet with a camera and a screen, with the first computer program and/or the second computer program and/or the third computer program and/or the fourth computer program being loaded onto the cell phone or tablet, preferably in the form of specialized software.

The device may comprise communication means for sending the updated images and/or video when the updated images are extracted from a video and/or a final model generated from the updated images at the end of the cycles of steps a) to d) and/or for receiving the reference representation before the first cycle.

The device may comprise a memory for storing updated images and/or video when the updated images are extracted from video and/or a final model generated from the updated images.

### EXAMPLE

A user wishes to obtain a model of at least one of his dental arches without having to visit a dental care professional, for example to have the state of his teeth checked. He launches a specialized software program, or “application”, on his cell phone or tablet, so that it can implement the method according to the invention.

Specialized software is considered to implement a step if it carries it out itself, or if it communicates with a remote computer for this purpose.

In the prior step, the specialized software can ask the user to acquire analysis images, preferably asking him to take some photos or a video, in order to determine characteristics of one of his dental arches, and select, from a database, a reference model in the form of a reference representation representing a reference arch with identical or similar characteristics. In particular, an analysis of the photos, possibly extracted from the video, can be used to determine the general shape of the arch. The reference model can be a typodont model with the same width as the user's arch, for example.

Alternatively, the reference model can be a model of a standard typodont usable for a category of users, or even a universal typodont, usable for any user.

The specialized software then displays the reference model on the cell phone screen and asks the user to take video of the arch.

In step a), in selfie mode and preferably without the use of a holder, the user takes video of his arch. Preferably, however, he uses a retractor, and in particular to spread the corners of the mouth to video the teeth at the back of his mouth and/or to spread the upper and lower lips to expose the incisors, preferably to the point of revealing the gums around them. Updated images are stills taken from the video, preferably at regular time intervals.

In step b), each updated video image is analyzed by specialized software to identify whether it represents a part of the arch. If necessary, the analysis can identify a part of the updated image, possibly in several pieces, which represents the dental arch, that is identify an “acquired 2D surface”.

The acquired 2D surface is then projected onto the reference model. To this end, the specialized software determines a direction in which to observe the reference model, so as to obtain a view that is substantially identical to the representation of the user's arch in the updated image. The acquired 2D surface is then projected onto the reference model in this direction, thus identifying a part of the reference model surface that represents the same parts of the reference arch as those of the user's arch represented by the acquired 2D surface.

This part of the reference model surface, or “labeled elementary region”, is then added to the labeled region of the reference model, which is the union of all elementary regions labeled in previous cycles. In this way, the specialized software can indicate at any time the unlabeled region, for which at least one updated image has yet to be taken and analyzed.

In this example, it is assumed that the goal is to cover the entire reference model, that is that the labeled region consists of the entire surface of the reference model, as shown in FIG. 3 at time tf. In one embodiment, however, the aim of the method is to cover only a portion of the surface of the reference model, and more generally of the reference representation. This portion can be identified on the reference model, for example by tracing its outline. Alternatively, only said portion can be displayed on the cell phone or tablet screen.

The specialized software then provides guide information so that the user can use the camera on the cell phone or tablet to reach the goal.

In a preferred embodiment, specialized software visually marks the labeled region, preferably by coloring it differently from the unlabeled region. The user immediately sees the unlabeled region and positions the camera accordingly. As coloring is in real time, the user receives immediate feedback on the relevance of the camera's instantaneous positioning, and can adapt it accordingly.

FIG. 3 shows different views of a reference model featuring a visual mark constituting guide information over time t, the reference model 20 being made up of tooth models 30 being progressively colored as a function of the image acquisitions made during the implementation of cycles of steps a) to d).

Before the first cycle, at time to, the labeled region of the reference model is the null set.

Each tooth model 30 can be colored when more than 80% of the surface of the tooth model is covered by the labeled region of the reference model, preferably when more than 90% of the surface of the tooth model is covered by the labeled region of the reference model, even better when the entire surface of the tooth model is covered by the labeled region of the reference model. The presentation of colored tooth models 32 provides guide information for the user.

A tooth model can be partially colored, the coloring corresponding to the surfaces of the tooth model covered by the labeled region of the reference model. In this way, the user can easily orient the image acquisition apparatus so as to acquire a surface of the tooth model not covered by the labeled region of the reference model. Other guide information can be provided, alternatively or preferably in addition to the visual labeling.

In one embodiment, the specialized software determines new “target” acquisition conditions for the camera, that is, for example, that would lead to the largest acquired 2D surface or the largest elemental labeled region, that is new acquisition conditions that are particularly effective in covering the surface of the reference model as quickly as possible. Preferably, the specialized software guides towards the new target acquisition conditions, for example by coloring a region of the reference model differently, or by displaying an arrow indicating a desired movement for the camera, or by emitting a sound signal that gets louder or sharper as the camera approaches the new target acquisition conditions.

When the goal is reached, for example because the entire surface of the reference model is covered by the labeled region, the stopping criterion is reached. A message can be sent to the user to inform them of this, so that they can end the video.

The specialized software then has enough updated images to generate an “updated” model of the user's arch. The final model can be generated directly from the updated images, like a 3D scan. It may result from the deformation of another model, for example the reference model, the deformation being guided so that the final model matches the updated images as closely as possible.

In the example shown in FIG. 3, at time tf all the tooth models are colored, indicating to the user that the last cycle of steps a) to d) has been completed and that the final step of generating a final three-dimensional model from the updated images can be carried out.

As is now clear, a device and a method according to the invention advantageously make it possible to increase the user's autonomy and improve the quality and content of images acquired by a user with no particular knowledge in the dental field. Advantageously, they enable a 3D model of a dental arch of a user to be produced remotely. This makes it much easier to determine orthodontic treatment at a distance, without the user having to make an appointment with a dental care professional.

Of course, the invention is not limited to the above-described and illustrated embodiments.

In particular, the invention is not limited by the use of the final model. This can be used, for example, to establish a diagnosis or an orthodontic treatment plan, or to manufacture an orthodontic aligner. In particular, the final model may represent only a region of interest for an analysis of the user's dental situation or for determining an orthodontic treatment, for example a region whose hygiene is to be assessed, a risk of recurrence in a particular area of the dental arch of a user, a region for which the user and/or a dental care professional wishes to program a possible aesthetic treatment.

The reference representation is not limited to a reference model. It can be a two-dimensional image, even if a model is preferred.

In a particular embodiment of the invention, step d) of the scanning

method is optional, the scanning method comprising:
a prior step of determining a model of a reference dental arch, referred to as the “reference representation”, then
a plurality of cycles of the following steps a) to c):
   a) acquisition, under acquisition conditions, of a two-dimensional image of said dental arch, or “updated image”, by means of an image acquisition apparatus, by the user;
   b) analysis of the updated image so as to identify the surface of the updated image representing the dental arch of a user, referred to as the “acquired 2D surface”, then outlining and labeling a surface of the reference representation that corresponds to the acquired 2D surface, referred to as the “elementary labeled region”, and adding the elementary labeled region to a “labeled region of the reference representation” that brings together all the elementary labeled regions defined during the previous cycles, which were empty prior to the first cycle;
   c) determination and presentation to the user of guide information for guiding the acquisition apparatus towards new acquisition conditions suitable for the next cycle to increase the labeled region of the reference representation;
   d) preferably, moving the acquisition apparatus to said new acquisition conditions; after the last cycle, the method includes a final step for generating a “final” three-dimensional model from said updated images.

Of course, the method may further comprise one or more of the optional features described above, which are compatible with an optional step d).

1. A method for scanning a dental arch of a user, the method comprising p1 a prior step of determining a model of a reference dental arch, referred to as the “reference representation”, then
a plurality of cycles of the following steps a) to c), preferably a) to d):
a) acquisition, under acquisition conditions, of a two-dimensional image of said dental arch, or “updated image”, by means of an image acquisition apparatus, by the user;
b) analysis of the updated image so as to identify the surface of the updated image representing the dental arch of a user, referred to as the “acquired 2D surface”, then outlining and labeling a surface of the reference representation that corresponds to the acquired 2D surface, referred to as the “elementary labeled region”, and adding the elementary labeled region to a “labeled region of the reference representation” that brings together all the elementary labeled regions defined during the previous cycles, which were empty prior to the first cycle;
c) determination and presentation to the user of guide information for guiding the acquisition apparatus towards new acquisition conditions suitable for the next cycle to increase the labeled region of the reference representation;
d) preferably, moving the acquisition apparatus to said new acquisition conditions;
after the last cycle, the method includes a final step for generating a “final” three-dimensional model from said updated images.

2. The method according to claim 1, wherein the updated images of successive cycles are extracted from a video acquired by the user via a tablet or a cell phone, said cycles being executed in real time during the acquisition of said video, at least a part of the guide information being presented on a screen of said tablet or said phone, respectively.

3. The method according to claim 1, wherein the guide information comprises a presentation of the labeled region on a screen of the image acquisition apparatus superimposed on the reference representation, wherein the unlabeled region has an appearance different from the labeled region.

4. The method according to claim 1, wherein the reference representation is:
one or more 3D or 2D illustrations of a dental arch, possibly photorealistic, or a “reference” model of a “reference” arch
representative of the “historic” arches of a set comprising more than 10 historic individuals, or
formed by assembling tooth models in an arrangement determined by analysis of one or more analysis images of the dental arch of the user.

5. The method according to claim 1, wherein, in step c) of any cycle, a view of the observed reference model is presented along an oriented direction of observation, with respect to the reference model, substantially like the direction of acquisition of the image updated in step a) of said cycle, with respect to the dental arch of the user, so that said view is substantially superimposable in register with said updated image.

6. The method according to claim 1, wherein, in any step c), the image acquisition apparatus provides variable information as a function of a difference between the actual acquisition conditions and said new acquisition conditions.

7. The method according to claim 1, wherein, for step a) of at least one cycle, the lips and/or cheeks of the dental arch of a user are moved apart so as to expose at least one tooth to the image acquisition apparatus.

8. The method according to claim 1, wherein, during the cycles, the following are acquired:
at least one updated image taken facing the user, at least one updated image taken to the right of the user and at least one updated image taken to the left of the user; and/or
at least one updated image taken facing the user and at least one updated image taken from above the user and at least one updated image taken from below the user; and/or
at least one updated image taken with the mouth open and at least one updated image taken with the mouth closed.

9. The method according to claim 1, wherein the user is more than 100 m from any dental care professional.

10. The method according to claim 1, wherein the cycles are stopped when more than 50% of the surface of the reference representation is represented on at least one updated image.

11. The method according to claim 1, wherein the final model is compared with another model representing the dental arch of a user and/or the final model is compared with the reference model, preferably by presenting this comparison to the user, for example by superimposing, preferably in register, the final model and the reference model.

12. The method according to claim 1, wherein the guide information comprises sound information, visual information, and/or sensitive information.

13. The method according to claim 1, wherein the reference representation is a model and the surface of the reference representation corresponding to the acquired 2D surface is determined
by searching for suitable virtual acquisition conditions for acquiring a view of the reference representation, referred to as the “reference image”, that best matches the image updated under said virtual acquisition conditions; then
by projecting the acquired 2D surface onto the reference representation, and along the direction of observation of said reference representation under said virtual acquisition conditions.

14. The method according to claim 13, wherein the virtual acquisition conditions are determined by seeking to superimpose noteworthy points of the dental arch represented on the updated image with the corresponding noteworthy points of the reference representation.

15. A device for carrying out a method according to claim , comprising
optionally, a first computer comprising a first computer program comprising program code instructions for generating the reference representation;
an image acquisition apparatus for carrying out steps a);
preferably a screen for displaying guide information during steps c);
a second computer, the same as or different from the first computer, comprising a second computer program comprising program code instructions for executing steps b) and c);
a third computer, the same as or different from the first and/or second computer(s), comprising a third computer program comprising program code instructions for implementing the final step of generating the final model of the dental arch of a user from the updated images;
preferably a fourth computer, the same as or different from the first and/or second and/or third computer(s), comprising a fourth computer program comprising program code instructions for comparing the final model with another model representing the dental arch of a user.
